(19) United States
(12) Patent Application Publication
(10) Pub. No.: US 2012/0004266 A1
(43) Pub. Date: Jan. 5, 2012

(54) DOPAMINE-BETA-HYDROXYLASE GENETIC POLYMORPHISM AND MIGRAINE

(75) Inventors: Lynette Robyn Griffiths, Queensland (AU); Rod A. Lea, Wellington (NZ); Francesca Fernandez, Singapore (SG)

(73) Assignee: Griffith University, Queensland (AU)

(21) Appl. No.: 12/999,707

(22) PCT Filed: Jun. 18, 2008
(86) PCT No.: PCT/AU08/00877

§ 371 Date: May 17, 2011

(51) Int. Cl. A61K 31/445; A61P 25/06; C12Q 1/68

(52) U.S. Cl. 514/327; 435/6.11

(57) ABSTRACT

The invention provides a method of determining whether or not an individual has a predisposition to migraine including the step of determining whether an isolated nucleic acid obtained from the individual comprises a nucleotide sequence corresponding to at least a fragment of a dopamine β-hydroxylase (DBH) gene promoter, wherein the presence of a −1021C→T single nucleotide polymorphism (SNP) in said nucleotide sequence indicates whether or not said individual has an increased predisposition to migraine compared to an individual without the polymorphism. DBH −1021C/C homozygotes are particularly susceptible to migraine. The −1021T allele may exert a protective effect. The method is particularly suited to detection of a predisposition to migraine with aura in females. The invention also provides a diagnostic kit for detecting a −1021C→T SNP associated with migraine. The method and kit may facilitate selection of individuals for migraine therapy which targets the dopaminergic system.

### FIELD OF THE INVENTION

THIS INVENTION relates to migraine. More particularly, this invention relates to identification of a polymorphism in the dopamine β hydroxylase gene that is associated with an increased genetic predisposition to migraine and uses thereof for detection of a genetic predisposition to migraine.

### BACKGROUND OF THE INVENTION

Migraine is a common neurological disorder that affects up to 25% of females and 8% of males in a western population (1). Migraine symptoms range from severe headache to nausea, vomiting, photophobia, phonophobia and variations of the visual field. The most common forms of this disorder have been classified as migraine with aura (MA) and migraine without aura (MO) (2).

Although definitive guidelines are available to classify headache, the aetiology of the disorder is less clear. Cerebral blood flow changes, specifically a decrease corresponding to the clinically affected area, have been noted as occurring before or at the onset of aura symptoms, in a number of sub-types of MA. In MO, however, regional cerebral blood flow remains normal or slightly increased. Several formative and perceptive sensory systems are part of these modulations, such as the autonomous nervous system, comprising notably by the sympathetic division (acting via noradrenaline, NA neurotransmitter), but also the diffuse modulatory system (with its transmitters serotonin, dopamine (DA), NA and acetylcholine) and/or the trigeminal sensory system (3-5). An imbalance in any of these neurological systems either at the transmitter or on the receptor side may lead to a higher susceptibility for migraine. NA is an element of the sympathetic system, involved in the regulation of vital body functions such as digestion, growth, immune response or energy storage. It increases for example heartbeat or, interestingly in regard to migraine, constricts blood vessels, modulates cerebrovascular autoregulation, reduces intracranial pressure, blood volume and cerebrospinal fluid production (6). In some migraineurs, the level of NA is significantly lower compared to migraine free subjects, possibly indicating a sympathetic hypofunction (7).

The catecholaminergic system, as part of the diffuse modulatory system of the brain, is involved in several functions affecting complex patterns such as mood, behaviour, attention, sleep-wake cycles, motor control, learning, brain metabolism and pain (8, 9). Numerous studies have implicated the catecholaminergic system in migraine (10). Several migraineurs have a hypersensitized dopaminergic system resulting among other things in an increased dopamine receptor density on T-cells (11, 12). The activated lymphocytes again, may be involved in the inflammatory processes during attacks (13). A central dopaminergic hyperfunction, and possible coexisting noradrenergic dysfunction, may lead to migraine attacks with severity positively correlated to dopamine concentration (14). Cerebral blood flow and somatosensory evoked potentials can also be changed by this dopamine hyperactivation (15).

The dopaminergic system has also been explored for a potential role in susceptibility to this complex neurological disorder. Several dopaminergic candidate genes have been investigated in different migraine case-control with varying results (16-18). Thirty years ago, a low level of Dopamine-beta-hydroxylase (DBH), intracellular enzyme catalysing the conversion of DA to NA, was observed in 3% (in adults) to 4% (in children) of the European population (19). The variation in both plasma DBH activity (20-22) and cerebrospinal-fluid levels of immunoreactive DBH protein (21) has then been shown as associated with several molecular markers at the DBH locus.

Two genetic markers (a 19 insertion/deletion (indel) and an STR) located in the promoter of the DBH gene (approximately 4.5 kilobases upstream of the transcriptional start site) and part of a 10 Kb block have been examined in an unrelated case-control population (150 cases vs 150 controls), but also in 263 patient from 82 families of migraineurs (25). The results showed a distortion of allele transmission of the short tandem repeat (STR) marker in individuals suffering from both migraine with or without aura (25). The first association between DBH alleles of this STR, and DBH plasma concentration was previously reported in a unrelated British population (20), an observation confirmed by Cubells et al. (21), who also investigated the promoter indel polymorphism. They showed that the indel marker was also functional, reporting that an individual with the deletion of both alleles had only half of the mean plasma enzyme activity compared with a homozygote with the insertion/insertion genotype (22).

Another investigation studying the indel marker in a larger Caucasian case-control population (275 cases vs 275 controls), reported a positive association between this 19 bp insertion/deletion (−4784-4803), and migraine (χ2=8.92, P=0.011) and more specifically with MA (χ2=11.48, P=0.003) (26).

### SUMMARY OF THE INVENTION

The present inventors have unexpectedly discovered a new genetic polymorphism in a promoter region of a human dopamine β-hydroxylase gene associated with, or linked to, a predisposition to migraine.

The present invention is therefore broadly directed to identification of a genetic predisposition to migraine according to the presence of a single nucleotide polymorphism in a promoter region of the human dopamine β-hydroxylase gene.

In a preferred form, the genetic polymorphism may also be associated with, or linked to, a level of human dopamine β-hydroxylase protein expression and/or enzymatic activity.

In a first aspect, the invention provides a method of determining whether or not an individual has a predisposition to migraine including the step of determining whether an isolated nucleic acid obtained from said individual comprises a nucleotide sequence corresponding to at least a fragment of a dopamine β-hydroxylase (DBH) gene promoter, wherein a single nucleotide polymorphism (SNP) in said nucleotide sequence indicates whether or not said individual has a predisposition to migraine.

Preferably, the SNP comprises a thymine or cytosine at position −1021 relative to the dopamine β-hydroxylase gene transcription start site.

Suitably, a cytosine at position −1021 is associated with a predisposition to migraine.

Preferably, a homozygote having a cytosine at position −1021 in both DBH alleles has a predisposition to migraine.

Suitably, a thymine at position −1021 is not associated with a predisposition to migraine or may be associated with a reduced likelihood of suffering from migraine.

Preferably, a homozygote having a thymine at position −1021 in both DBH alleles has a reduced likelihood of suffering from migraine.

In a second aspect, the invention provides a kit for use in the method of the aforementioned aspect, said kit comprising one or more primers, probes and, optionally, one or more other reagents for identifying said dopamine β hydroxylase gene promoter SNP.

In a particular embodiment, the kit comprises one or more primers for nucleic acid sequence amplification of a nucleotide sequence corresponding to at least a fragment of a dopamine β-hydroxylase gene promoter that comprises nucleotide −1021.

The kit may further comprise a HhaI restriction endonuclease.

In a third aspect, the invention provides a method of treating migraine including the steps of:
(i) selecting an individual comprising a single nucleotide polymorphism (SNP) in a dopamine β-hydroxylase (DBH) gene promoter which is associated with a predisposition to migraine;
(ii) treating the individual to thereby at least alleviate one or more symptoms of migraine.

Preferably, the SNP comprises a cytosine at position −1021 relative to the dopamine β-hydroxylase gene transcription start site.

More preferably, the individual is a homozygote having a cytosine at position −1021 in both DBH alleles,

Suitably, according to the aforementioned aspects, said individual is a male or female human.

Preferably, the individual is a female.

Preferably, migraine is migraine with aura (MA).

Throughout this specification, unless the context requires otherwise, the words “comprise”, “comprises” and “comprising” will be understood to imply the inclusion of a stated integer or group of integers but not the exclusion of any other integer or group of integers.

### DETAILED DESCRIPTION OF THE PREFERRED EMBODIMENT

The present invention is at least partly based on results of an analysis of dopamine β hydroxylase (DBH) gene markers in two independent populations of 200 cases and 200 matched controls and the second population of 300 migraineurs and 300 controls. Two functional SNPs were examined in two independent large case-control populations, one within the promoter of DBH gene (−1021C→T, rs1611115) and another SNP (+1603C/T, rs6271) in exon 11 of the same gene, encoding a non-conservative difference in primary amino acid sequence (arg535cys) in two independent large case-control populations

The results showed a significant association for both allelic and genotypic frequency distribution between the DBH marker in the promoter (−1021C→T) and migraine in the first (P=0.004 and P=0.012 respectively) and the second (P=0.013 and P=0.031 respectively) tested populations. In addition, these positive results have also been found between this functional marker and migraine with aura (MA) subtype in both studied populations (P<0.05). In contrast, there was no significant association between either genotype and/or allelic frequencies for the DBH marker located in the exon (+C1603T) and migraine, respectively in the first analysed population and also in the second population (P≧0.05).

The present invention therefore has arisen from the finding that the DBH gene −1021C→T SNP is a unique, DBH gene promoter SNP that indicates whether an individual has a genetic predisposition to migraine.

Typically, a cytosine at position −1021 is associated with a predisposition to migraine.

Preferably, a homozygote individual having a cytosine at position −1021 in both alleles (a −1021C/C genotype) has a genetic predisposition to migraine.

Typically, a thymine at position −1021 is not associated with a predisposition to migraine, or is associated with a reduced probability of suffering from migraine.

Preferably, a homozygote individual having a thymine at position −1021 in both alleles (i.e a −1021T/T genotype) has a reduced probability of suffering from migraine.

Furthermore, the −1021T allele in heterozygotes may confer a protective effect, thereby indicating a reduced probability of suffering from migraine.

In another embodiment, the −1021C allele appears to correlate with decreased levels of DBH enzyme activity, which suggests that the −1021C→T SNP is the first “functional” DBH promoter polymorphism that correlates with DBH enzyme activity. Accordingly, migraineurs harbouring the −1021C allele (particularly in homozygotes) may be selected as particularly suited to treatment with therapeutic agents that target the dopaminergic system.

A further embodiment of the present invention arises from the observation that a −1021C/C homozygous genotype may be associated with reduced risk of developing emesis and/or risk of presenting with diarrhea related symptoms of migraine.

Throughout this specification “predisposed and predisposition” in the context of migraine means that an individual is susceptible to, or has an increased likelihood or probability of, suffering from migraine and includes situations where said individual is not yet exhibiting clinical symptoms of migraine and where said individual is already displaying migraine symptoms.

As used herein, “migraine” includes “migraine with aura” (MA) and “migraine without aura” (MO).

Preferably, migraine is migraine with aura (MA).

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native or recombinant form.

The term “nucleic acid” as used herein designates single- or double-stranded mRNA, RNA, cRNA and DNA inclusive of cDNA and genomic DNA and DNA-RNA hybrids.

A “polynucleotide” is a nucleic acid having eighty (80) or more contiguous nucleotides, while an “oligonucleotide” has less than eighty (80) contiguous nucleotides.

A “probe” may be a single or double-stranded oligonucleotide or polynucleotide, suitably labeled for the purpose of detecting complementary sequences in Northern or Southern blotting, for example.

A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™. Non-limiting examples of primers useful according to the invention comprise respective nucleotide sequences set forth in SEQ ID NO:1 and SEQ ID NO:2. However, it will be readily appreciated by persons skilled in the art that the human DBH gene sequence may be used as the basis for designing alternative primers that allow amplification of a fragment of a DBH gene promoter comprising nucleotide −1021.

The terms “anneal”, “hybridize” and “hybridization” are used herein in relation to the formation of bimolecular complexes by base-pairing between complementary or partly-complementary nucleic acids in the sense commonly understood in the art. It should also be understood that these terms encompass base-pairing between modified purines and pyrimidines (for example, inosine, methylinosine and methyladenosine) and modified pyrimidines (for example thiouridine and methylcytosine) as well as between A, G, C, T and U purines and pyrimidines. Factors that influence hybridization such as temperature, ionic strength, duration and denaturing agents are well understood in the art, although a useful operational discussion of hybridization is provided in to Chapter 2 of CURRENT PROTOCOLS IN MOLECULAR BIOLOGY (Eds. Ausubel et al. John Wiley & Sons NY, 2000), particularly at sections 2.9 and 2.10.

The term “gene” is used herein as a discrete nucleic acid unit or region that may comprise one or more of introns, exons, open reading frames, splice sites and regulatory sequences such as promoters and polyadenylation sequences.

The term “single nucleotide polymorphism (SNP)” is used herein to indicate any nucleotide sequence variation in an allelic form of a gene that occurs in a human population. This term encompasses mutation, insertion, deletion and other like terms that indicate specific types of SNPs.

In the context of the present invention by “corresponds to” and “corresponding to” is meant that an isolated nucleic acid comprises a nucleotide sequence that is, or is complementary to, a nucleotide sequence of at least a fragment of a DBH gene promoter comprising nucleotide −1021.

In one embodiment, the present invention provides for determination of a predisposition to migraine according to whether an individual has −1021C and/or −1021T DBH alleles. Position −1021 is located in a promoter region of a human DBH gene 5′ of the normal transcription start site. It will therefore be appreciated that by isolating a nucleic acid corresponding to at least a fragment of a DBH gene promoter comprising nucleotide −1021, a determination can be made as to whether an individual is predisposed to migraine.

Varying DBH protein and/or enzymatic activity levels have been postulated as being involved in the migraine process with an increase of dopamine, resulting from a lower DBH activity, shown to be positively correlated with migraine severity. The −1021 DBH SNP is in linkage disequilibrium with the previously reported migraine associated DBH microsatellite. Both polymorphism are functional, significantly affecting DBH enzyme activity. It is thus plausible that the −1021C allele is a functional variant that may contribute to a migraine predisposition by way of lower or reduced levels of DBH protein and/or enzymatic activity.

By “protein” is meant an amino acid polymer. The amino acids may be natural or non-natural amino acids, D- or L-amino acids as are well understood in the art.

A “peptide” is a protein having less than fifty (50) amino acids.

A “polypeptide” is a protein having fifty (50) or more amino acids.

Accordingly, the invention contemplates measurement of DBH protein and/or enzymatic activity levels in addition to methods that identify a DBH promoter SNP at position −1021.

The method may be used independently of clinical diagnosis or may be used in conjunction therewith to confirm or assist clinical diagnosis of migraine, inclusive of migraine with aura and migraine without aura.

Furthermore, the method of the invention may be used in combination with methods that identify other genetic polymorphisms associated with migraine. One non-limiting example is a method which identified polymorphisms in female steroid sex hormone receptor genes, as described in International Publication WO2005/026385 or an indel marker as hereinbefore described.

Generally, the methods of the invention are nucleic acid-based methods, given that the DBH SNP described herein is a non-coding polymorphism that does not affect an amino acid sequence of a DBH protein.

However, as hereinbefore described, the invention also contemplates measurement of DBH protein and/or enzymatic activity levels in addition to methods that identify a DBH promoter SNP at position −1021.

Such DBH protein detection methods are well known in the art and include western blotting, ELISA, two dimensional protein profiling, protein arrays, immunoprecipitation, radioimmunoassays and radioligand binding and DBH enzyme activity assays, although without limitation thereto.

Protein-based methods typically, although not exclusively, include the step of obtaining a serum sample for measurement of DBH protein and/or enzymatic activity levels.

Nucleic acid-based methods may include the step of obtaining said isolated nucleic acid from said individual.

An isolated nucleic acid corresponding to at least a fragment of a DBH gene promoter may be obtained from any appropriate human source of nucleic acid, such as lymphocytes or any other nucleated cell type, preferably obtainable by a minimally-invasive method.

The at least a fragment of the isolated nucleic acid may be in the form of genomic DNA, RNA or cDNA reverse-transcribed from isolated RNA.

It will be appreciated that according to the invention, nucleic acid fragments of a DBH gene promoter comprising nucleotide −1021, or a corresponding isolated nucleic acid, suitably comprise less than 100% of the promoter.

Typically, in certain embodiments fragments may have at least 9, 15, 20, 50 or up to 80 contiguous nucleotides (such as oligonucleotide primers and probes).

In other embodiments, fragments may have 80, 100, 150, 200, 300, 500 or more contiguous nucleotides (such as PCR amplification products).

In a particular embodiment of the invention, a fragment may be a product of nucleic acid sequence amplification.

A non-limiting example of such fragments is a 131 bp fragment comprising nucleotide −1021 of a DBH gene promoter.

Non-limiting examples of primers suitable for nucleic acid sequence amplification comprise respective nucleotide sequences according to SEQ ID NO:1 and SEQ ID NO:2.

However, it will be readily appreciated by persons skilled in the art that the human DBH gene may be used as the basis for designing alternative primers that allow amplification of a fragment of a DBH gene promoter comprising nucleotide −1021.

In this regard, it will be appreciated that preferred diagnostic methods employ a nucleic acid sequence amplification technique.

Suitable nucleic acid amplification techniques are well known to the skilled addressee, and include polymerase chain reaction (PCR) and ligase chain reaction (LCR) as for example described in Chapter 15 of Ausubel et al. supra; strand displacement amplification (SDA) as for example described in U.S. Pat. No. 5,422,252; rolling circle replication (RCR) as for example described in Liu et al., 1996, J. Am. Chem. Soc. 118 1587 and International application WO 92/01813, and Lizardi et al., (International Application WO 97/19193); nucleic acid sequence-based amplification (NASBA) as for example described by Sooknanan et al., 1994, Biotechniques 17 1077; ligase chain reaction (LCR) as for example described in International Application WO89/09385; Q-β replicase amplification as for example described by Tyagi et al., 1996, Proc. Natl. Acad. Sci. USA 93 5395; and helicase-dependent amplification as for example described in International Publication WO 2004/02025.

As used herein, an “amplification product” is a nucleic acid produced by a nucleic acid sequence amplification technique.

A preferred nucleic acid sequence amplification technique is PCR.

Notwithstanding the foregoing, the invention contemplates other nucleic acid detection methods that may be useful for detecting the DBH gene promoter SNP polymorphism.

For example, a PCR method that may also be useful is Bi-PASA (Bidirectional PCR Amplification of Specific Alleles), as for example described in Liu et al. 1997, Genome Res. 7 389-399.

Another potentially useful PCR method as allele-specification oligonucleotide hybridization, as for example described in Aitken et al., 1999, J Natl Cancer Inst 91 446-452.

It will also be well understood by the skilled person that identification of the DBH gene promoter SNP may be performed using any of a variety of techniques such as fluorescence-based melt curve analysis, SSCP analysis, denaturing gradient gel electrophoresis (DGGE), restriction endonuclease digestion or direct sequencing of amplification products.

Melt curve analysis can be performed using fluorochrome-labeled allele-specific probes which form base-pair mismatches when annealing to wild-type DNA strands in heterozygotes. Alternatively, fluorescent DNA-intercalating dyes such as SYBR Green 1 can reveal the presence of these base-pair mismatches by virtue of their lower melting temperature (Tₘ) compared to fully complementary sequences. A useful example of allele-specific melt curve analysis can be found, for example, in International Publication No. WO97/46714.

DGGE also exploits Tₘ differences, but uses differential electrophoretic migration through gradient gels as a means of distinguishing subtle nucleotide sequence differences between alleles. Examples of DGGE methods can be found in Fodde & Losekoot, 1994, Hum. Mutat. 3 83-9 and U.S. Pat. Nos. 5,045,450 and 5,190,856.

The DBH gene SNP used according to the invention may be identified by direct sequencing of a PCR amplification product, for example. An example of nucleic acid sequencing technology is provided in Chapter 7 of CURRENT PROTOCOLS IN MOLECULAR BIOLOGY Eds. Ausubel et al. (John Wiley & Sons NY USA 1995-2001).

In yet another embodiment, mass spectroscopy (such as MALDI-TOF) may be used to identify nucleic acid polymorphisms according to mass. In a preferred form, such methods employ mass spectroscopic analysis of primer extension products, such as using the MassARRAY™ technology of Sequenom.

In a further embodiment, said DBH gene promoter SNP may be identified by a microarray method of the invention.

Microarray technology has become well known in the art and examples of methods applicable to microarray technology are provided in Chapter 22 of CURRENT PROTOCOLS IN MOLECULAR BIOLOGY Eds. Ausubel et al. (John Wiley & Sons NY USA 1995-2001).

With respect to the present invention, a preferred microarray format comprises a substrate such as a glass slide or chip having an immobilized, ordered grid of a plurality of nucleic acid molecules, such as cDNA molecules, although without limitation thereto.

A microarray would typically comprise a nucleic acid having said estrogen receptor gene polymorphism and/or a nucleic acid having said progesterone receptor gene polymorphism together with control estrogen receptor and progesterone receptor nucleic acids.

Such a microarray could also include a plurality of other nucleic acids indicative of other diseases that have an underlying genetic basis and be useful in large scale genetic screening, for example.

With regard to restriction endonuclease digestion of amplification products, it will be appreciated that −1021T alleles do not digest with HhaI restriction endonuclease whereas −1021C alleles may be digested to give 109 bp and 22 bp fragments.

It will also be appreciated that the method of the invention also extends to a method of analysis of one or more gene sequence databases to identify one or more individuals having a DBH gene promoter SNP as herein described.

In this regard, an increasing aspect of molecular medicine is the establishment of computer-searchable databases that comprise genetic information obtained from patients, which databases may readily be interrogated to correlate the presence of a DBH gene promoter SNP, as herein described, with genetic information obtained from a particular patient.

It will also be appreciated from the foregoing that the invention contemplates a kit for molecular genetic detection of a predisposition to migraine.

In a particular embodiment, the kit comprises
(a) primers for nucleic acid sequence amplification of at least a fragment of a DBH gene promoter comprising nucleotide −1021; and
(b) a HhaI restriction endonuclease.

Non-limiting examples of primers in (a) comprise respective nucleotide sequences according to SEQ ID NO:1 and SEQ ID NO:2.

One or more other reagents are contemplated such as probes for hybridization-based methods and detection reagents useful in enzymatic, colorimetric and/or radionuclide-based detection of nucleic acids, although without limitation thereto.

In another aspect, the invention provides a method of treating migraine including the steps of:
(I) selecting an individual comprising a single nucleotide polymorphism (SNP) in a dopamine β-hydroxylase gene promoter which is associated with a predisposition to migraine;
(II) treating the individual to thereby at least alleviate one or more symptoms of migraine.

Suitably, the SNP comprises a cytosine at position −1021 relative to the dopamine β-hydroxylase gene transcription start site.

Preferably, the individual is a −1021C/C homozygote.

Preferably, migraine is migraine with aura (MA).

Preferably, the individual is a female human.

It will therefore be appreciated that the invention may facilitate treatment of migraine by identifying individuals that have a functional DBH gene promoter SNP that is associated with reduced levels of DBH enzyme activity and elevated levels of dopamine. In this regard, the diagnostic method of the invention may be useful in selecting individuals responsive to therapeutic agents that restore DBH enzyme activity and/or inhibit dopamine or dopamine receptor activity (e.g. dopamine receptor agonists such as haloperidol).

So that the present invention may be more readily understood and put into practical effect, the skilled person is referred to the following non-limiting examples.

### EXAMPLES

### Methods

### Subjects:

The study protocol was approved by Griffith University's ethics committee for experimentation on humans. All individuals were of Caucasian origin and gave informed consent before participating in the research. Migraineurs were diagnosed as having either MA or MO, based strictly on criteria specified by the International Headache Society (IHS) (3). All individuals were grouped together and phenotyped as being affected with typical migraine (MA+MO=Migraine), as well as being examined separately as MA or MO subgroups. The first study population was comprised of 200 migraineurs and 200 unrelated control individuals, and the second one contains 300 migraineurs and 300 controls. To minimise potential bias from population stratification, the control group was matched for sex, age (+/−5 years) and ethnicity (as previously described (26).

### Markers/Genotyping:

The study investigated two different polymorphisms at the DBH gene locus, one within the promoter and the other one in the coding region. The first marker was SNP located at −1021 bp to the translational start site of the DBH gene (ref SNP database, rs 1611115), named DBHpr. The PCR analysis was carried out using a modification of a previously described method (24, 28). The PCR reactions (10 μl final volume) contained 2 mmol/L MgCl₂, 0.8 mol/L of each primer, 200 mol/L dNTPs, 1 unit of Taq polymerase and approximately 20 μg of genomic DNA.

Primers were:

(SEQ ID NO: 1)
Sense: 5′-GGAGGGACAGCT TCT AGTCC-3′
(SEQ ID NO: 2)
Anti sense: 5′-CACCTCTCCCTCCTGTCCTCTCGC-3′.

Thermal cycling was performed with an initial denaturation of 5 minutes at 94° C., followed by 35 cycles of 30 sec at 94° C., 30 sec at 60° C., 30 sec at 72° C., and a terminal extension of 10 min at 72° C. The PCR products were digested with HhaI and analyzed by electrophoresis on 3% agarose gels. Ethidium bromide stained gels were digitally imaged and manually scored for genotypes. The PCR products were 131 bp in size. The T alleles did not digest with HhaI, whereas C alleles digested to give 109 bp and 22 bp fragments.

The second marker was SNP located at +1603 bp in the coding region of the DBH gene (ref SNP database, rs6271), named DBHex. The PCR analysis was also performed using a modification of a previously described study (24, 28). The PCR reactions (10 μl final volume) contained 2 mmol/L MgCl₂, 0.8 mol/L of each primer, 200 mol/L dNTPs, 1 unit of Taq polymerase and approximately 20 μg of genomic DNA.

Primers were:

(SEQ ID NO: 3)
Sense: 5′-CCAGGGACAGGACTCGAGTTG-3′
(SEQ ID NO: 4)
Anti sense: 5′-AGCAGTTTGGAGTGCAGACCC-3′.

Thermal cycling was performed with an initial denaturation of 5 minutes at 94° C., followed by 35 cycles of 30 sec at 94° C., 30 sec at 62° C., 30 sec at 72° C., and a final extension of 10 min at 72° C. The PCR products were digested with Bst UI and analyzed by electrophoresis on 3% agarose gels. Ethidium bromide stained gels were digitally imaged and manually scored for genotypes. The PCR products were 352 bp in size. The T alleles did not digest with Bst UI, whereas C alleles digested to give 3 fragments of 184, 139 and 29 bp.

The genotyping for the DBHpr marker has also been performed in some samples from the first population for corroboration of obtained results using the High Resolution Melt (HRM) methods. HRM was carried out using a modification of previous reports of this technique [HRM, Liew], using Rotor-Gene 6000 (HRM)™ (Corbett Research). The same forward and reverse primers for DBHpr that were used above (RLFP method) were also used for HRM as they efficiently amplified a short size PCR product of 131 bp. PCR reactions contained 1 μl of genomic DNA, 0.2 mmol/L MgCl₂, 0.2 mmol/L dNTPs, 1.25 unit of Platinum Taq DNA polymerase 300 nM of each primer and 1.5 μM of SYTO 9 (Invitrogen) made up to 25 μl with filter sterilized water. Samples were run on a Rotor-Gene 6000 (HRM)™ (Corbett Research) using temperature cycling conditions of: 10 minutes at 95° C. followed by 40 cycles of 95° C. for 5 seconds and 60° C. for 10 seconds. This was followed by a melt step of 65-85° C. in 0.2° C. increments pausing for 2 seconds per step. The increase in SYTO 9 fluorescence was monitored in real time during the PCR and the subsequent decrease during the melt phase by acquiring each cycle/step to the green channel (470 nm excitation and 510 nm emission) of the Rotor-Gene. Genotypes were scored by examining normalized and difference melt plots using the Rotor-Gene Software.

### Statistical Analysis

To detect association between each marker and migraine, we performed chi-square (χ²) analysis to test for significant differences in allele and genotype frequencies in case versus control results (29). χ² provides the likelihood of a deviation in the distribution of the same attributes in different classes (e.g. allelic frequencies in controls versus affected subjects). If the probability (P-volume) of an equal distribution between the two groups is below a determined significance level α (0.05), the statistical output will show enough significance to assume LD and therefore association.

We performed χ² analysis for migraineurs MA, MO and combined migraine groups versus control subjects for the DBHpr and DBHex polymorphisms. We also tested for linkage disequilibrium between biallelic tested markers using the Graphical Overview of Linkage Disequilibrium (GOLD) program, a new bioinformatic software to analyse dense genetic maps. In addition, the GOLD program provides a distinct graphical representation of disequilibriums patterns (30). For this analysis, we included data found our previous genotyping of the insertion/deletion marker (19 bp), localised at −4784 bp within promoter of DBH gene and reported to be significantly associated with migraine (26).

Results were also tested for Hardy-Weinberg Equilibrium (HWE) investigating genotype frequencies of the DBHpr and DBHex markers to detect a deviation from the normal genotype distribution in the population and odds ratios were calculated to assess the magnitude of associations. We also performed endophenotype analysis, investigating the distribution of several migraine associated symptoms, including nausea, vomiting and diarrhea, according to genotype.

### RESULTS

Two markers, one located at 1.02 kb upstream of the starting point and the other one at +1.6 Kb in the coding of the DBH gene, were analysed for association with migraine two independent populations (200 migraineurs versus 200 healthy individuals; 300 migraineurs versus 300 healthy individuals respectively) of Australian Caucasians. Genotypes for both DBH markers were determined in the migraine case and control populations. The distribution of DBHpr and DBHex genotypes in the studied population did not deviate significantly from Hardy-Weinberg Equilibrium (P>0.05).

The results of the allelic and genotypic frequency distribution of DBHpr in the two studied populations were analysed.

The data are provided in Tables 1-4.

Results showed a significant association of DBHpr alleles with migraine in the first and the second independent analysed populations (χ²=8.24, P=0.004; χ²=6.17, P=0.013).

This positive result was also found for the genotypic frequencies of the DBHpr marker (χ²=8.73, 2df, P=0.012 and χ²=6.91, 2df, P=0.031 respectively). In addition, this significant association was also observed in the MA group in the first and the second studied populations for both genotypic (χ²=6.57, 2df, P=0.037; χ²=7.58, 2df, P=0.022 respectively) and allelic frequencies (χ²=6.26, 2df, P=0.011; χ²=7.19, 2df, P=0.007, respectively).

Subjects with two copies of the allele T genotype had a decreased risk of migraine compared to controls in both tested populations (OR=0.55, 95% Cl 0.37-0.83 and OR=0.67, 95% Cl 0.48-0.92).

In regard to the analyses by gender for DBHpr, a significant association was found for all combinated migraine compared to controls in females for both genotypic (χ²=8.56, 2df, P=0.013) and allelic frequencies (χ²=7.88, P=0.005), but was not significant in male groups (χ²=0.79, 2df, P=0.067; χ²=0.8, P=0.37 respectively) in the first population. The identical pattern was observed for the genotypic frequencies (χ²=10.32, 2df, P=0.006) and allelic frequencies (χ²=7.88, P=0.005) in the female group of the second studied population. However, no significant association was found in the male group of the second population for both genotypic (χ²=0.57, 2df, P=0.75) and allelic frequencies (χ²=0.26, P=0.6).

There was no significant association between either genotype or allelic frequencies for DBHex and migraine (χ²=2.95, 2df, P=0.229; χ²=2.44, P=0.118 respectively) in the first analysed population but also in the second population (χ²=2.31, 2df, P=0.315; χ²=0.93, P=0.335). When we analysed by gender and by subtype of migraine, no significant association was similarly observed for DBHex genotype and allelic distribution (P>0.05) in both studied populations.

LD was calculated for the present studied DBH genetic markers, including the insertion/deletion (Indel) reported associated with both migraine (χ²=8.92, 2df, P=0.011) and more specifically with MA (χ²=11.48, 2df, P=0.003) groups in our previous study (26). The analysis of LD between the studied genetic markers revealed a moderate but significant linkage between DBHpr and Indel (D′=0.42, P=0.00001). However, this LD value was found to be non-significant (P<0.05) when LD was measured between DBHex and DBHpr on one hand, and DBHex and indel on the other hand. Our previous association between DBH markers located in the promoter and migraine (and more specifically MA) has been confirmed in this study and extended to the DBHpr marker, in linkage with the DBH Indel marker.

Endophenotype analysis of the positively associated DBHpr marker was also undertaken. We were particularly interested in nausea and emesis as dopamine receptor antagonists are an established class of anti-emetic agent [34] and diarrhoea, as dopaminergic defects have been associated with enteric dysfunction in humans [35]. The results of this analysis showed that 8% of individuals with the CC genotype suffered from diarrhoea compared to 23% of individuals with the CT/TT genotype. Migraineurs with at least one T allele were 3 times more likely to suffer diarrhoea. Another interesting finding was that individuals with at least one T allele were also more likely to suffer from emesis (60% CC genotype compared to 78% with CT/TT genotype). Hence it appears that possession of the CC genotype may confer a protective effect for both emesis and diarrhoea associated with migraine.

### DISCUSSION

During the last three decades, the dopaminergic system has been considered as playing a part in the pathogenesis of migraine. Numerous studies have reported a genetic association between migraine and several polymorphism in DA genes, e.g. with the D2 dopaminergic receptor (10, 16) and D4 dopaminergic receptor (17). Dopamine Beta Hydroxylase enzyme plays also an important role in the regulation of the DA levels in the synapse. Interestingly significant differences in serum DBH have been observed in migraine patients compared with healthy control subjects (31, 32) and during a migraine attack (33). Several functional polymorphisms have been reported for the DBH gene. Lea and colleagues have examined the prevalence of different alleles of both markers, the DBH STR and DBH insertion/deletion in an association study with 177 unrelated migraineurs and 182 controls plus a TDT analysis of 296 subjects (263 affected) from 82 families of migraineurs (25). The results showed a distortion of allele transmission of the microsatellite (STR) marker in individuals suffering from both migraine with or without aura (25). Our previous study undertaken in a larger case-control population has also reported a positive association between the deletion genotype and migraine (p≦0.05), and also migraine with aura (p≦0.01) (26).

In the present study, we examined the distribution of genotype and allelic frequencies of two functional polymorphisms of the DBH gene, DBHpr located at 1.02 kb upstream of the starting point and DBHex in the exon 11 of the DBH gene in two independent and unrelated case-control populations.

The analysis for both allelic and genotypic frequency distribution showed a significant association between the DBHpr marker and migraine in the first (P=0.004 and P=0.012 respectively) and the second (P=0.013 and P=0.031 respectively) independent tested populations. These positive results have also been found between the DBHpr marker and the MA subtype in both studied populations (P<0.05). Obtained genotypic frequencies for the DBHpr marker (genotypes: TT=3-6%, TC=32-36% and CC=60-62%) in our control groups in both tested populations gave results similar to previous studies (34-36). Healy and collaborators investigating the role of −C1021T polymorphism in Parkinson Disease sufferers compared to two large independent cohorts of controls (n=637 for cohort A and n=450 for cohort B), showed a genotypic profile comparable to our results found in our control populations (genotypes: TT=6.3%, TC=32.3% and CC=61.2% for cohort A and TT=5.8%, TC=31.3% and CC=62.8% for cohort B) (36). The same group of researchers has also reported no association for the DBHpr polymorphism between a population suffering for epilepsy compared to the identical control cohorts (A and B) (34). Tang and collaborators have recently examined the relationship between DBH polymorphisms (including DBHpr and DBHex polymorphisms) and plasma DBH activity in an African-American population (35). Genotypic frequencies (DBHpr and DBHex) reported in this study (genotypes: TT=7.3%, TC=25.7% and CC=67% and TT-, TC=5.5% and CC=94.5% respectively) again showed a similar pattern to that observed in our first (genotypes: TT=5.7%, TC=32.4% and CC=61.9% for DBHpr and TT-, TC=12.4% and CC=87.6% for DBHex markers) and second (genotypes: TT=3.6%, TC=35.8% and CC=60.6% for DBHpr and TT-, TC=12.4% and CC=87.6% for DBHex markers) tested populations. DBHex was not significantly associated with migraine or its subtypes (MA, MO) with chi-square results producing P values greater than 0.05 for most analyses in both studied populations. There was no substantial LD between DBHpr and DBHex polymorphisms as measured by r2 (r2<0.001), and as previously reported by Zabetian et al. (2001) (23) and more recently by Tang and collaborators (35).

The activity of the DBH enzyme can be measured in serum (or the plasma) due to the release of this enzyme from the central and peripheral adrenergic and noradrenergic neurons as well as adrenomedullar cells during an excitation of the sympathetic system (37). Several polymorphisms of the DBH gene have been reported in previous studies as being associated with the activity of plasma DBH (21, 22, 24, 27, 28, 38). The DBH promoter variant DBHpr, is responsible for 31-52% of the variance of the plasma DBH enzyme activity in Caucasian population (24). Interestingly, the DBH exon 11 polymorphism also tested in our study seems to independently account for additional variance in plasma DBH activity. Recently, Tang et. al. have evaluated the effect of four DBH polymorphisms (DBHpr, indel, rs 2519152, DBHex) on the activity of plasma DBH in African American populations (35). This report showed that a low activity DBH profile is significantly associated with haplotypes T-C-C (for DBHpr-rs 2519152-DBHex respectively) (P=0.0036), but also with haplotype C-T-C (P=0.0025) (35). Significant differences of serum DBH activity have been observed in migraine (MO and MA) patients compared with healthy control subjects (31). As regards as the lower percentage of migraineurs with T allele (1.2%) for DBHpr marker compared to the controls (3.5-5.7%) observed in our examined populations, it would be judicious to test the rs 2519152 polymorphism in our samples in the aim to confirm that migraine sufferers with C allele for DBHpr and DBHex express also the T allele for rs2519152, verifying the C-T-C haplotype, reported in Tang and coll. study (35).

Dopamine beta hydroxylase plays a key part in the balance of NA/DA circulating levels in the synaptic space and is available to act on postsynaptic neurons. A wide variety of clinical signs indicate that dysfunction of the sympathetic system exists in migraine sufferers, in both in the interictal and attacks periods (39). Many independent investigations have reported lower levels of supine plasma NA (51% to 53%) compared to controls, indicating a hypoactivity of the sympathetic nervous system (7, 32, 40, 41). Opposed to the hypofunction of the sympathetic nervous system, many studies reported a hyperactivity of the dopaminergic system as indicated by a high levels of dopamine in plasma measured during attacks (42), but also between attacks in both plasma and platelets in migraineurs compared to controls (43). In addition, clinical trials involving treatment with dopamine receptor antagonists (44, 45) confirms the involvement of dopamine in migraine. In fact, administration of dopaminergic agonists can induce the same symptoms seen during a migraine attack and dopamine antagonist treatment can be used effectively in migraineurs during an attack (44, 45). Hypersensitivity of the dopaminergic system can also lead to vegetative symptoms such as nausea, sweating, yawning, observed also during migraine attacks. In the present study, we investigated the association of specific endophenotypes (as emesis and diarrhea) in relation to the genotypes of DBHpr analysed in our populations. Interestingly, the CT/TT combined genotype group showed significantly more than two times the risk to develop emesis (OR 2.3 (95% CI 0.886 to 5.792)) and three times the risk to present with diarrhea related symptoms (OR 3.14 (95% CI 0.9908 to 9.9286)) compared to the CC genotype group for DBHpr marker.

This study showed a significant association between the DBHpr polymorphism and migraine, more specifically with migraine with aura in two independent populations, which confirms that the DBH gene is a good candidate gene for migraine susceptibility. Although the DBHpr polymorphism has a functional role and has been shown to be responsible for 31% to 52% of the variance of plasmatic DBH, it accounts for only half of the total variability of the enzyme and other factors may be involved in this variation. Further analysis regarding other functional polymorphisms of this gene and how they affect the DBH enzyme and more specifically, impact on migraine, are warranted.

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

All computer programs, algorithms, patent and scientific literature referred to in this specification is incorporated herein by reference in their entirety.

TABLE 1
Distribution of the DBH pr polymorphism (SNP ref rs 1611115) in DBH gene in migraineurs
and controls of original sample (MO migraine without aura, MA migraine with aura) in the
first studied population.

| | N | Alleles | | Genotypes | | |
|---|---|---|---|---|---|---|
| Group | (alleles) | T | C | TT | CT | CC |
| Migraine | 340 | 46 (13.5%) | 294 (86.5%) | 2 (1.2%) | 42 (24.7%) | 126 (74.1%) |
| Male | 86 | 13 (15.1%) | 73 (84.9%) | 1 (2.3%) | 11 (25.6%) | 31 (72.1%) |
| Female | 254 | 33 (13%) | 221 (87%) | 1 (0.8%) | 31 (24.4%) | 95 (74.8%) |
| MA | 180 | 23 (12.8%) | 157 (87.2%) | 1 (1.1%) | 21 (23.3%) | 69 (75.6%) |
| MO | 160 | 23 (14.4%) | 137 (85.6%) | 1 (1.3%) | 21 (26.2%) | 57 (72.5%) |
| Control | 352 | 77 (21.9%) | 275 (78.1%) | 10 (5.7%) | 57 (32.4%) | 109 (61.9%) |
| Male | 94 | 19 (20.2%) | 75 (79.8%) | 2 (4.3%) | 15 (31.9%) | 30 (63.8%) |
| Female | 258 | 58 (22.5%) | 200 (77.5%) | 8 (6.2%) | 42 (32.6%) | 79 (61.2%) |

| | | |
|---|---|---|
| Total Case Vs | χ² = 8.24 p = 0.004 | χ² = 8.73 p = 0.012 |
| Control | | |

TABLE 2
Distribution of the DBHpr polymorphism (SNP ref rs 1611115) in DBH gene in migraineurs
and controls of original sample (MO migraine without aura, MA migraine with aura) in the
second population.

| | N | Alleles | | Genotypes | | |
|---|---|---|---|---|---|---|
| Group | (alleles) | T | C | TT | CT | CC |
| Migraine | 490 | 76 (15.5%) | 414 (84.5%) | 3 (1.2%) | 70 (28.6%) | 172 (70.2%) |
| Male | 70 | 13 (18.6%) | 57 (81.4%) | 2 (5.7%) | 9 (25.7%) | 24 (68.6%) |
| Female | 480 | 63 (15%) | 357 (85%) | 1 (0.5%) | 61 (29%) | 148 (70.5%) |
| MA | 408 | 60 (14.7%) | 348 (85.3%) | 3 (1.5%) | 54 (26.5%) | 147 (72%) |
| MO | 82 | 16 (19.5%) | 66 (80.5%) | 0 | 16 (39%) | 25 (61%) |
| Control | 558 | 120 (21.5%) | 438 (78.5%) | 10 (3.6%) | 100 (35.8%) | 169 (60.6%) |
| Male | 84 | 13 (15.5%) | 71 (84.5%) | 1 (2.4%) | 11 (26.2%) | 30 (71.4%) |
| Female | 474 | 107 (22.6%) | 367 (77.4%) | 9 (3.8%) | 89 (37.6%) | 139 (58.6%) |

| | | |
|---|---|---|
| Total Case Vs | χ² = 6.17 p = 0.013 | χ² = 6.91 p = 0.031 |
| Control | | |

TABLE 3
Chi-squared (χ²) analysis of the genotypic frequencies in all
Migraine Groups against Controls for the DBHpr polymorphism
in the two studied populations

| | Population 1 | | Population 2 | |
|---|---|---|---|---|
| | χ² Value | P value | χ² Value | P value |
| Migraine vs controls | 8.73 | 0.012 | 6.91 | 0.031 |
| MA vs controls | 6.57 | 0.037 | 7.58 | 0.022 |
| MO vs controls | 3.94 | 0.13 | 1.57 | 0.45 |
| Female Migraine vs Female | 8.56 | 0.013 | 10.32 | 0.006 |
| controls | | | | |
| Male Migraine vs Male | 0.79 | 0.67 | 0.57 | 0.75 |
| controls | | | | |

TABLE 4
Chi-squared (χ²) analysis of the allele frequencies in all
Migraine Groups against Controls for the DBHpr polymorphism
in the two studied populations

| | Population 1 | | Population 2 | |
|---|---|---|---|---|
| | χ² Value | P value | χ² Value | P value |
| Migraine vs controls | 8.24 | 0.004 | 6.17 | 0.013 |
| MA vs controls | 6.26 | 0.011 | 7.19 | 0.007 |
| MO vs controls | 3.94 | 0.047 | 0.17 | 0.68 |
| Female Migraine vs Female | 7.88 | 0.005 | 8.29 | 0.004 |
| controls | | | | |
| Male Migraine vs Male | 0.8 | 0.37 | 0.26 | 0.6 |
| controls | | | | |

### REFERENCES

1. Launer L J, Terwindt G M, Ferrari M D. The prevalence and characteristics of migraine in a population-based cohort: the GEM study. Neurology. 1999 Aug. 11; 53 (3):537-42.
2. HCCIHS. Headache Classification Committee for the International Headache Society. Classification and diagnostic criteria for headache disorders, cranial neuralgias and facial pain 2nd edition. Cephalgia. 2004; 24 (Suppl 1):1-60.
3. Friberg L, Olesen J, Iversen H K, Sperling B. Migraine pain associated with middle cerebral artery dilatation: reversal by sumatriptan. Lancet. 1991 Jul. 6; 338(8758):13-7.
4. O'Connor T P, van der Kooy D. Enrichment of a vasoactive neuropeptide (calcitonin gene related peptide) in the trigeminal sensory projection to the intracranial arteries. J Neurosci. 1988 July; 8(7):2468-76.
5. Suzuki N, Hardebo J E. The cerebrovascular parasympathetic innervation. Cerebrovasc Brain Metab Rev. 1993 Spring; 5(1):33-46.
6. Esler M, Rumantir M, Kaye D, Jennings G, Hastings J, Socratous F, et al. Sympathetic nerve biology in essential hypertension. Clin Exp Pharmacol Physiol. 2001 December; 28(12):986-9.
7. Martinez F, Castillo J, Rodriguez J R, Leira R, Noya M. Neuroexcitatory amino acid levels in plasma and cerebrospinal fluid during migraine attacks. Cephalalgia. 1993 April; 13(2):89-93.
8. Mitler M M, Erman M, Hajdukovic R. The treatment of excessive somnolence with stimulant drugs. Sleep. 1993 April; 16(3):203-6.
9. Mochizucki D. Serotonin and noradrenaline reuptake inhibitors in animal models of pain. Hum Psychopharmacol. 2004 October; 19 Suppl 1:S15-9.
10. Peroutka S J. Dopamine and migraine. Neurology. 1997 September; 49(3):650-6.
11. Barbanti P, Fabbrini G, Ricci A, Bruno G, Cerbo R, Bronzetti E, et al. Reduced density of dopamine D2-like receptors on peripheral blood lymphocytes in Alzheimer's disease. Mech Ageing Dev. 2000 Dec. 1; 120(1-3):65-75.
12. Cerbo R, Barbanti P, Buzzi M G, Fabbrini G, Brusa L, Roberti C, et al. Dopamine hypersensitivity in migraine: role of the apomorphine test. Clin Neuropharmacol. 1997 February; 20(1):36-41.
13. Levite M, Chowers Y, Ganor Y, Besser M, Hershkovits R, Cahalon L. Dopamine interacts directly with its D3 and D2 receptors on normal human T cells, and activates beta1 integrin function. Eur J Immunol. 2001 December; 31(12):3504-12.
14. Castillo J, Martinez F, Suarez C, Naveiro J, Lema M, Noya M. Cerebrospinal fluid tyrosine and 3,4-dihydroxyphenylacetic acid levels in migraine patients. Cephalalgia. 1996 February; 16(1):56-61.
15. Fanciullacci M, Alessandri M, Del Rosso A. Dopamine involvement in the migraine attack. Funct Neurol. 2000; 15 Suppl 3:171-81.
16. Del Zompo M, Cherchi A, Palmas M A, Ponti M, Bocchetta A, Gessa G L, et al. Association between dopamine receptor genes and migraine without aura in a Sardinian sample. Neurology. 1998 September; 51(3):781-6.
17. Mochi M, Cevoli S, Cortelli P, Pierangeli G, Soriani S, Scapoli C, et al. A genetic association study of migraine with dopamine receptor 4, dopamine transporter and dopamine-beta-hydroxylase genes. Neurol Sci. 2003 February; 23(6):301-5.
18. Peroutka S J, Wilhoit T, Jones K. Clinical susceptibility to migraine with aura is modified by dopamine D2 receptor (DRD2) NcoI alleles. Neurology. 1997 July; 49(1):201-6.
19. Weinshilboum R M. Serum dopamine beta-hydroxylase. Pharmacol Rev. 1978 June; 30(2):133-66.
20. Wei J, Ramchand C N, Hemmings G P. Possible control of dopamine beta-hydroxylase via a codominant mechanism associated with the polymorphic (GT)n repeat at its gene locus in healthy individuals. Hum Genet. 1997 January; 99(1):52-5.
21. Cubells J F, van Kammen D P, Kelley M E, Anderson G M, O'Connor D T, Price L H, et al. Dopamine beta-hydroxylase: two polymorphisms in linkage disequilibrium at the structural gene DBH associate with biochemical phenotypic variation. Hum Genet. 1998 May; 102(5):533-40.
22. Cubells J F, Kranzler H R, McCance-Katz E, Anderson G M, Malison R T, Price L H, et al. A haplotype at the DBH locus, associated with low plasma dopamine beta-hydroxylase activity, also associates with cocaine-induced paranoia. Mol Psychiatry. 2000 January; 5(1):56-63.
23. Zabetian C P, Anderson G M, Buxbaum S G, Elston R C, Ichinose H, Nagatsu T, et al. A quantitative-trait analysis of human plasma-dopamine beta-hydroxylase activity: evidence for a major functional polymorphism at the DBH locus. Am J Hum Genet. 2001 February; 68(2):515-22.
24. Zabetian C P, Buxbaum S G, Elston R C, Kohnke M D, Anderson G M, Gelernter J, et al. The structure of linkage disequilibrium at the DBH locus strongly influences the magnitude of association between diallelic markers and plasma dopamine beta-hydroxylase activity. Am J Hum Genet. 2003 June; 72(6):1389-400.
25. Lea R A, Dohy A, Jordan K, Quinlan S, Brimage P J, Griffiths L R. Evidence for allelic association of the dopamine beta-hydroxylase gene (DBH) with susceptibility to typical migraine. Neurogenetics. 2000 September; 3(1):35-40.
26. Fernandez F, Lea R A, Colson N J, Bellis C, Quinlan S, Griffiths L R. Association between a 19 bp deletion polymorphism at the dopamine beta-hydroxylase (DBH) locus and migraine with aura. J Neurol Sci. 2006 Dec. 21; 251(1-2):118-23.
27. Cubells J F, Zabetian C P. Human genetics of plasma dopamine beta-hydroxylase activity: applications to research in psychiatry and neurology. Psychopharmacology (Berl). 2004 August; 174(4):463-76.
28. Tang Y, Anderson G M, Zabetian C P, Kohnke M D, Cubells J F. Haplotype-controlled analysis of the association of a non-synonymous single nucleotide polymorphism at DBH (+1603C-->T) with plasma dopamine beta-hydroxylase activity. Am J Med Genet B Neuropsychiatr Genet. 2005 Nov. 5; 139(1):88-90.
29. Nahmias J, Burley M W, Povey S, Porter C, Craig I, Wolfe J. A 19 bp deletion polymorphism adjacent to a dinucleotide repeat polymorphism at the human dopamine beta-hydroxylase locus. Hum Mol Genet. 1992 July; 1(4):286.
30. Abecasis G R, Cookson W O. GOLD—graphical overview of linkage disequilibrium. Bioinformatics. 2000 February; 16(2):182-3.
31. Gallai V, Gaiti A, Sarchielli P, Coata G, Trequattrini A, Paciaroni M. Evidence for an altered dopamine beta-hydroxylase activity in migraine and tension-type headache. Acta Neurol Scand. 1992 October; 86(4):403-6.
32. Gotoh F, Kanda T, Sakai F, Yamamoto M, Takeoka T. Serum dopamine-beta-hydroxylase activity in migraine. Arch Neurol. 1976 September; 33(9):656-7.
33. Anthony M. Biochemical indices of sympathetic activity in migraine. Cephalalgia. 1981 June; 1 (2): 83-9.
34. Depondt C, Cock H R, Healy D G, Burley M W, Weinshenker D, Wood N W, et al. The −1021C->T DBH gene variant is not associated with epilepsy or antiepileptic drug response. Neurology. 2004 Oct. 26; 63(8):1497-9.
35. Tang Y L, Epstein M P, Anderson G M, Zabetian C P, Cubells J F. Genotypic and haplotypic associations of the DBH gene with plasma dopamine beta-hydroxylase activity in African Americans. Eur J Hum Genet. 2007 August; 15(8):878-83.
36. Healy D G, Abou-Sleiman P M, Ozawa T, Lees A J, Bhatia K, Ahmadi K R, et al. A functional polymorphism regulating dopamine beta-hydroxylase influences against Parkinson's disease. Ann Neurol. 2004 March; 55(3):443-6.
37. O'Connor D T, Cervenka J H, Stone R A, Levine G L, Parmer R J, Franco-Bourland R E, et al. Dopamine beta-hydroxylase immunoreactivity in human cerebrospinal fluid: properties, relationship to central noradrenergic neuronal activity and variation in Parkinson's disease and congenital dopamine beta-hydroxylase deficiency. Clin Sci (Lond). 1994 February; 86(2):149-58.
38. Cubells J F, Price L H, Meyers B S, Anderson G M, Zabetian C P, Alexopoulos G S, et al. Genotype-controlled analysis of plasma dopamine beta-hydroxylase activity in psychotic unipolar major depression. Biol Psychiatry. 2002 Mar. 1; 51(5):358-64.
39. Peroutka. S J. Migraine: a chronic sympathetic nervous system disorder. Headache. 2004 January; 44(1):53-64.
40. Mikamo K, Takeshima T, Takahashi K. Cardiovascular sympathetic hypofunction in muscle contraction headache and migraine. Headache. 1989 February; 29(2): 86-9.
41. Takeshima T, Takao Y, Urakami K, Nishikawa S, Takahashi K. Muscle contraction headache and migraine. Platelet activation and plasma norepinephrine during the cold pressor test. Cephalalgia. 1989 March; 9(1):7-13.
42. Pradalier A, Launay J M, Soliman M, Dreux C, Guittard M, Hanna K M, et al. [Platelet release of dopamine in the common migraine attack]. Presse Med. 1987 July 11-18;16(27):1321-3.
43. D'Andrea G, Granella F, Perini F, Farruggio A, Leone M, Bussone G. Platelet levels of dopamine are increased in migraine and cluster headache. Headache. 2006 April; 46(4):585-91.
44. Honkaniemi J, Liimatainen S, Rainesalo S, Sulavuori S. Haloperidol in the acute treatment of migraine: a randomized, double-blind, placebo-controlled study. Headache. 2006 May; 46(5):781-7.
45. Fisher H. Migraine research methods. Cmaj. 1997 Oct. 15; 157(8):1015-6, 8.

1. A method of determining whether or not an individual has a predisposition to migraine including the step of determining whether an isolated nucleic acid obtained from said individual comprises a nucleotide sequence corresponding to at least a fragment of a dopamine β-hydroxylase (DBH) gene promoter, wherein a single nucleotide polymorphism (SNP) in said nucleotide sequence indicates whether or not said individual has a predisposition to migraine.

2. The method of claim 1, wherein the SNP is at a position corresponding to nucleotide −1021 of a human DBH gene.

3. The method of claim 2, wherein the SNP comprises a cytosine at position −1021 which indicates that said individual has a predisposition to migraine.

4. The method of claim 3, wherein the individual is a homozygote.

5. The method of claim 2, wherein the SNP comprises a thymine at position −1021 which indicates that said individual does not have a predisposition to migraine or has a reduced likelihood of suffering from migraine.

6. The method of claim 5, wherein the individual is a homoozygote.

7. The method of claim 1, wherein the isolated nucleic acid is an amplification fragment obtained from said individual by PCR.

8. The method of claim 7, wherein PCR is performed using primers that respectively comprise a nucleotide sequence according to SEQ ID NO:1 and SEQ ID NO:2.

9. The method of claim 7, wherein said amplification fragment is subjected to restriction endonuclease digestion.

10. The method of claim 9, wherein restriction endonuclease digestion is performed using HhaI restriction endonuclease.

11. The method of claim 1, further including the step of measuring a level of DBH protein and/or DBH enzymatic activity, wherein a relatively reduced level and/or activity is indicative of a predisposition to migraine.

12. The method of claim 1, wherein migraine is migraine with aura (MA).

13. The method of claim 1, wherein the individual is a female human.

14. The method of claim 1, wherein said SNP indicates whether or not said individual has a predisposition to one or more migraine symptoms selected from emesis and diarrhea, or is less likely to display one or more migraine-associated symptoms selected from emesis and diarrhea.

15. The method of claim 1, which includes analysis of one or more gene sequence databases comprising genetic information obtained from said individual to thereby identify whether or not said individual has a predisposition to migraine.

16. A kit for use in the method of claim 1, said kit comprising one or more primers, probes and, optionally, one or more other reagents for identifying said SNP.

17. The kit of claim 16, which comprises one or more primers for nucleic acid sequence amplification of a nucleotide sequence corresponding to at least a fragment of a dopamine β hydroxylase gene promoter that comprises nucleotide −1021.

18. The kit of claim 17, wherein the primers comprise a nucleotide sequence according to SEQ ID NO:1 and SEQ ID NO:2.

19. The kit of claim 18, further comprising a restriction endonuclease.

20. The kit of claim 19, wherein the restriction endonuclease is HhaI.

21. A method of treating migraine including the steps of:
(i) selecting an individual comprising a single nucleotide polymorphism (SNP) in a dopamine β-hydroxylase (DBH) gene promoter which is associated with a predisposition to migraine compared to an individual without the polymorphism; and
(ii) treating the individual to thereby at least alleviate one or more symptoms of migraine.

22. The method of claim 21, wherein the SNP is at a position corresponding to −1021 of a human DBH gene.

23. The method of claim 22, wherein the SNP comprises a cytosine at position −1021.

24. The method of claim 23, wherein the individual is a homozygote.

25. The method of claim 21, further including the step of measuring a level of DBH protein and/or DBH enzymatic activity before step (ii), wherein a relatively reduced level and/or activity is indicative of a predisposition to migraine.

26. The method of claim 25, wherein step (ii) includes administering a dopamine receptor antagonist to the individual.

27. The method of claim 21, wherein migraine is migraine with aura (MA).

28. The method of claim 21, wherein the individual is a female human.


SEQUENCE LISTING (4 sequences)

SEQ ID NO: 1 - DNA, 20 bp, Artificial sequence
  DBH sense primer 1
ggagggacagcttctagtcc

SEQ ID NO: 2 - DNA, 24 bp, Artificial sequence
  DBH antisense primer 1
cacctctccctcctgtcctctcgc

SEQ ID NO: 3 - DNA, 21 bp, Artificial sequence
  DBH sense primer 2
ccagggacaggactcgagttg

SEQ ID NO: 4 - DNA, 21 bp, Artificial sequence
  DBH antisense primer 2
agcagtttggagtgcagaccc